(19) United States
(12) Patent Application Publication
(10) Pub. No.: US 2017/0007625 A1
(43) Pub. Date: Jan. 12, 2017

(54) NOVEL FORMULATIONS TO INHIBIT CYCLOOXYGENASE AND PRO-INFLAMMATORY CYTOKINE MEDIATED DISEASES

(71) Applicant: Bioved Pharmaceuticals, Inc., San Jose, CA (US)

(72) Inventors: DEEPA CHITRE, LOS GATOS, CA (US); DEBENDRANATH DEY, FREMONT, CA (US)

(73) Assignee: BIOVED PHARMACEUTICALS, INC., San Jose, CA (US)

(21) Appl. No.: 14/627,652

(22) Filed: Feb. 20, 2015

Related U.S. Application Data
(63) Continuation of application No. 12/632,824, filed on Dec. 8, 2009, now abandoned.
(60) Provisional application No. 61/201,647, filed on Dec. 11, 2008.

(51) Int. Cl. A61K 31/573; A61K 31/4402; A61K 31/415

(52) U.S. Cl. CPC A61K 31/573; A61K 31/415; A61K 31/4402

(57) ABSTRACT

The invention provides method and composition for alleviating one or more symptoms associated with a medical condition mediated by one or more of a cyclooxygenase, a pro-inflammatory cytokine, and a pro-inflammatory enzyme. The method includes administering an effective amount of a composition to a person suffering from the medical condition. The composition essentially includes a set of plant extracts. The set of plant extracts include an extract of Withania somnifera, an extract of Boswellia serrata, an extract of Curcuma longa, and an extract of Zingiber officinale. Wherein, one or more extracts of the set of plant extracts includes one or more desired active ingredient in an amount greater than an amount of other active ingredients present in the one or more extracts. The composition can also be used as combination therapy with any other known anti-inflammatory agents.

### RELATED APPLICATIONS

This continuation application claims the benefit of priority to copending U.S. patent application Ser. No. 12/632,824 filed Dec. 8, 2009, which in turn claims the benefit of priority to U.S. Provisional Patent Application No. 61/201,647 filed Dec. 11, 2008, both incorporated herein by reference in their entirety.

### FIELD OF THE INVENTION

The present invention generally relates to a composition and a method for treating one or more medical conditions mediated by one or more of, a cyclooxygenase, pro-inflammatory cytokine, and pro-inflammatory enzymes.

### BACKGROUND OF THE INVENTION

There is a tremendous surge in knowledge regarding pathological mediators like pro-inflammatory cytokines like Tumor Necrosis Factor Alpha (TNF-.alpha.), anti-inflammatory cytokines, pro-inflammatory enzymes like cyclooxygenases (COXs), phosphodiesterases (PDEs), and inducible Nitric Oxide Synthetase (iNOS). The role of the pathological mediators in different medical conditions is evolving day-by-day thereby revealing new insights into the pathogenesis of inflammatory diseases. This in turn is revolutionizing the management of patients with inflammatory diseases mediated by the pathological mediators.

There are several diseases that are mediated by the pathological mediators like pro-inflammatory cytokines and pro-inflammatory enzymes. These diseases include, for example, Ankylosing Spondylitis (AS), Psoriasis, Rheumatoid Arthritis (RA), Osteoarthritis (OA), Inflammatory Bowel Disease (IBD), Crohn's Disease (CD) Multiple Sclerosis (MS), Alzheimer's Disease (AD), Chronic Obstructive Pulmonary Disease (COPD), and Behchet's Disease (BD).

Ankylosing Spondylitis (AS):

The treatment of AS generally includes individualized physical therapy and Non-Steroidal Anti-Inflammatory Drugs (NSAIDs). However, the NSAIDs are associated with gastrointestinal side effects and therefore additional gastro-protective therapy is required. Further, evidence to date does not currently support the use of Disease Modifying Anti-Rheumatic Drugs (DMARDs), corticosteroids, or radiotherapy in AS. Newer therapies, such as the TNF inhibitors, have transformed the treatment paradigm in AS, especially for those patients with aggressive disease. However, TNF inhibitors produce a range of cellular responses, wherein the cellular responses may include cell death, survival, differentiation, proliferation and migration. For example, vascular endothelial cells respond to TNF by undergoing a number of pro-inflammatory changes. These pro-inflammatory changes lead to increase in leukocyte adhesion, trans-endothelial migration, and vascular leak and promote thrombosis.

Psoriasis:

The first-line of treatment of psoriasis includes topical agents like corticosteroids, calcipotriene, and tazarotene. The second line of treatment includes phototherapy with ultraviolet B or photo-chemotherapy with psoralens plus ultraviolet A (PUVA). The third line of treatment includes systemic anti-psoriatic drugs like methotrexate, acetretin, and cyclosporin. However, the serious side-effects associated with the topical agents, the phototherapy and especially with systemic agents like methotrexate and acetretin will be well appreciated by a person skilled in the art. Further, the systemic anti-psoriatic drugs like acetretin are immuno-modulatory and cannot be given to a patient for long term.

Behcet's Disease (BD):

Treatment of BD is challenging, and has to be tailored to the pattern of organ involvement for each patient and often requires combination therapies. In general, topical treatment of BD using corticosteroids with or without antibiotics is helpful in controlling oral and genital ulcers. Immuno-suppressive drugs have been shown to be moderately successful in inducing and maintaining remissions. Systemic corticosteroids, once widely used in BD, are now reserved only for the most severe cases of inflammatory eye disease and vasculitis. The corticosteroids like azathioprine, methotrexate, mycophenolate mofetil, cyclosporine, tacrolimus, cyclophosphamide, and chlorambucil are recommended in intra-ocular inflammation associated with BD. New therapies with limited experience include the TNF-.alpha. inhibitors, interferon alpha, monoclonal antibodies against lymphocyte surface antigens, intravenous immunoglobulin (IVIG), and the intraocular delivery of immuno-suppressive agents. However, the side-effects associated with the therapies involving corticosteroids and immuno-suppressive agents will be well appreciated by a person skilled in the art.

Inflammatory Bowel Disease (IBD) and Crohn's Disease (CD):

Significant current therapeutic strategies in maintaining remission in CD include 5-aminosalicylates (e.g. sulfasalazine, mesalazine), thiopurines (e.g. azathioprine, 6-mercaptopurine [mercaptopurine]), methotrexate and infliximab. However, 5-aminosalicylates have efficacy limited to either surgically induced remission and/or small bowel CD. The immuno-modulators have an established role in maintaining remission in CD. For example, Azathioprine and 6-mercaptopurine are effective in chronic active CD and corticosteroid-dependent CD. Methotrexate has similar indications as that of azathioprine and 6-mercaptopurine. However, methotrexate is used only as an alternative in patients who are intolerant of, or resistant to, thiopurines.

In addition, Biologic Response Modifiers (BRMs) or Biologics are used successfully for treating patients with IBD. However, the biologics and BRMs are injectables and are very expensive as compared to conventional drugs. Further, evidence of risks like infection and malignancy that are associated with the biologics is also mounting, thereby limiting their use.

Rheumatoid Arthritis (RA):

The mainstay in treatment of RA includes anti-inflammatory agents such as corticosteroids, NSAIDs such as Ibuprofen, Indomethacin, Diclofenac, Piroxicam, and the like and COX inhibitors such as Celecoxib, Rofecoxib, Etoricoxib, and the like. However, the anti-inflammatory agents have a limited activity. Further, the manifestations of RA become resistant to anti-inflammatory therapy over a period ranging from weeks to months. In addition, the anti-inflammatory agents are associated with significant side effects for example, gastro-intestinal side effects, because of which the anti-inflammatory agents are not used for a long term. Over the last 10 years, there has been consistent use of methotrexate in the treatment of RA. However, methotrexate is a potentially toxic agent. Additionally, biologics, BRMs, and anti-IL-6 receptor monoclonal antibodies are recently been used in the treatment of RA. However, the biologics and BRMs are associated with a significant risk of developing serious infections and a dose dependent risk of developing malignancies. Hence, there is a need for a safe and effective long-term therapy in RA.

Osteoarthritis (OA):

There is a dearth of therapeutic agents with proven efficacy and minimal toxicity that can control or arrest the relentless progression of OA and lifelong pain in majority of patients with OA. Thus, there is a need for safe and effective therapeutic agents for treating OA that besides being anti-inflammatory can provide chondro-protection.

Chronic Obstructive Pulmonary Disease (COPD):

Current drug treatment of COPD improves symptoms but does not alter the underlying progression of COPD. Further, PDE-4 inhibitors have been recently shown to document clinical efficacy in the treatment of COPDs like asthma, allergy and inflammatory pulmonary diseases. However, use of PDE-4 inhibitor in COPD is hampered due to side-effects like nausea, emesis and diarrhea that are associated with PDE-4 inhibitors.

Thus, the current treatments of inflammatory diseases like, AS, Psoriasis, BD, IBD, CD, RA, OA, and COPD include use of NSAIDs, DMARDs, biologics, immuno-modulating agents, immuno-suppressive agents, inhibitors of cytokines, and inhibitors of pro-inflammatory enzymes. However, the current treatments of inflammatory diseases are associated with a range of side-effects and thus cannot be used for long-term treatment. Therefore, there is need in the art for therapeutic agents of proven efficacy and minimal toxicity that can control or arrest the relentless progression of inflammatory diseases. Further, there is need in the art for safe long-term therapy of inflammatory diseases.

### BRIEF DESCRIPTION OF THE FIGURES

The accompanying figures, incorporated in and form part of the specification, serve to illustrate various examples in accordance with the present invention.

FIG. 1 illustrates a comparison between anti-inflammatory activities of Vehicle, Prednisolone, Formulation A, and Formulation B in carrageenan-induced paw edema in rats.

FIG. 2 illustrates changes in the volume of the exudative fluid in the vehicle and in animals treated with Ibuprofen and Formulation B.

FIG. 3 illustrates changes in the weight of dried granuloma pouches in the vehicle and in animals treated with Ibuprofen and Formulation B.

FIG. 4 illustrates paw thickness in animals as a percent of the paw thickness at day 0 during the Adjuvant Induced Arthritis study in animals.

FIG. 5 illustrates percent body weight gain in animals during the Adjuvant Induced Arthritis study in animals.

FIG. 6 illustrates the clotting time in seconds in the animals (male) over a period of 180 days of toxicity study using Formulation B.

FIG. 7 illustrates the clotting time in seconds in the animals (Female) over a period of 180 days of toxicity study using Formulation B.

FIG. 8 illustrates the percent Hematocrit Volumes of the animals (Male) over a period of 180 days of toxicity study using Formulation B.

FIG. 9 illustrates the percent Hematocrit Volumes of the animals (Female) over a period of 180 days of toxicity study using Formulation B.

FIG. 10 illustrates the anti-inflammatory activities of Formulation B; Celecoxib; Celecoxib in combination with Formulation B; Chondroitin; and Chondroitin in combination with Formulation B.

FIG. 11 illustrates production of TNF-.alpha. in the mouse macrophage cells activated by LPS and treated separately with Dexamethasone and different doses of Formulation B.

FIG. 12 illustrates production of NO in the mouse macrophage cells activated by LPS and treated separately with Dexamethasone and different doses of Formulation B.

FIG. 13 illustrates effectiveness of Formulation B and Sulfasalazine in inhibiting DSS induced colitis, wherein the inhibition of DSS induced colitis is measured as improvement in body weight loss in animals.

### DETAILED DESCRIPTION OF THE INVENTION

Before describing in detail, the embodiments that are in accordance with the present invention, it should be observed that the embodiments reside primarily in combinations of method steps and constituents related to method and composition for alleviating one or more symptoms associated with a medical condition mediated by one or more of a cyclooxygenase, a pro-inflammatory cytokine, and a pro-inflammatory enzyme. Accordingly, the composition and method steps have been represented, showing only those specific details that are pertinent to understanding the embodiments of the present invention so as not to obscure the disclosure with details that will be readily apparent to those of ordinary skill in the art.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or composition. An element proceeded by “comprises . . . a” does not, without more constraints, preclude the existence of additional identical elements in the process, method, article, or composition that comprises the element. In this document, the terms, “Cyclo-oxygenase”, Cyclooxygenase, Cycloxygenase and COX are used synonymously, unless and until specified.

Generally speaking, pursuant to various embodiments, the invention provides a method and a composition for alleviating one or more symptoms associated with a medical condition mediated by one or more of a cyclooxygenase, a pro-inflammatory cytokine, and a pro-inflammatory enzyme. The method includes, administering to a person suffering from the medical condition an effective amount of the composition. The composition includes a set of plant extracts. The set of plant extracts includes an extract of Withania somnifera, an extract of Boswellia serrata, an extract of Curcuma longa, and an extract of Zingiber officinale. The set of plant extracts includes one or more extracts, wherein the one or more extracts includes one or more desired active ingredients in an amount that is greater than an amount of other active ingredients present in the one or more extracts. For example, the one or more extracts may include the extract of Withania somnifera and the one or more desired active ingredients may include Withanolide-D. The amount of Withanolide-D present in the extract of Withania somnifera is greater than the amount of other active ingredients present in the extract of Withania somnifera.

The one or more symptoms include, for example, but not limited to pain and inflammation. The one or more medical conditions include, for example, but are not limited to, a medical condition that is mediated by one or more pathological mediators. The one or more pathological mediators include, for example, but not limited to, a cyclooxygenase, a pro-inflammatory cytokine, and a pro-inflammatory enzyme. The cyclooxygenase may include one or more of, but are not limited to, cyclooxygenase-1 (COX1) and cyclooxygenase-2 (COX-2). The pro-inflammatory cytokine may include one or more of, but are not limited to, interleukin-6 (IL-6), interleukin-1 (IL-1) and Tumor Necrosis Factor-alpha (TNF-.alpha.). The pro-inflammatory enzyme may include one or more of, for example, but not limited to, phosphodiesterase-4 (PDE-4) and Inducible Nitric Oxide Synthetase (iNOS).

Further, the medical condition may include one or more of, but are not limited to, Rheumatoid Arthritis (RA), Osteoarthritis (OA), Inflammatory Bowel Disease (IBD), Crohn's Disease (CD), psoriasis, Ankylosing Spondylitis (AS), Behcet's Disease (BD), Multiple Sclerosis (MS), Alzheimer's Disease (AD), Chronic Obstructive Pulmonary Disease (COPD), an airway inflammation, type-II diabetes mellitus, atherosclerosis, obesity and restenosis. It will be appreciated by a person skilled in the art that one or more symptoms associated with any medical condition that is mediated by the one or more pathological mediators disclosed herein can be alleviated by using the composition and the method disclosed herein, without deviating from the scope of the invention.

In an exemplary embodiment, the method includes administering to the person suffering from the medical condition an effective amount of the composition. The composition includes the set of plant extracts. The set of plant extracts includes, for example, but not limited to, 10% to 75% of the extract of Withania somnifera, 10% to 75% of the extract of Boswellia serrata, 3% to 30% of the extract of Curcuma longa, and 3% to 25% extract of Zingiber officinale. More preferably, the set of plant extracts includes 30% to 50% of the extract of Withania somnifera, 30% to 50% of the extract of Boswellia serrata, 5% to 15% of the extract of Curcuma longa, and 3% to 13% extract of Zingiber officinale. Still more preferably, the set of plant extracts includes the extract of Withania somnifera, the extract of Boswellia serrata, the extract of Curcuma longa, and the extract of Zingiber officinale in a concentration ratio of 1:1:0.25:0.20 respectively.

In accordance with various embodiments, the composition includes a capsule. The capsule includes 30 mg to 300 mg of the extract of Withania somnifera, 30 mg to 300 mg of the extract of Boswellia serrata, 5 mg to 50 mg of the extract of Curcuma longa, and 4 mg to 40 mg of the extract of Zingiber officinale. In an exemplary embodiment, the capsule includes 20 mg to 120 mg of the extract of Withania somnifera, 20 mg to 120 mg of the extract of Boswellia serrata, 15 mg to 30 mg of the extract of Curcuma longa, and 15 mg to 25 mg of the extract of Zingiber officinale.

In accordance with various embodiments, the extract of Withania somnifera includes 0.05% to 2.5% of Withanolide-D on a dry basis. The amount of the Withanolide-D present in the extract of Withania somnifera is greater than the amount of any other active ingredient present in the extract of Withania somnifera. Further, the pharmacological activity and efficacy of the extract of Withania somnifera present in the composition towards alleviating the one or more symptom associated with the medical condition is attributed to the amount of the Withanolide-D present in the extract of Withania somnifera.

The plant-based formulations offer a distinct advantage over steroids and NSAIDS in terms of non-drug dependence and lack of gastric irritation and other side effects.

The extract of Boswellia serrata includes 30% to 60% of total boswellic acids on a dry basis. The amount of total boswellic acids present in the extract of Boswellia serrata is greater than the amount of any other active ingredient present in the extract of Boswellia serrata. Further, the pharmacological activity and efficacy of the extract of Boswellia serrata present in the composition towards alleviating the one or more symptoms associated with the medical condition is attributed to the amount of total boswellic acids present in the extract of Boswellia serrata.

The extract of Curcuma longa includes 20% to 50% of curcuminoids on a dry basis. The amount of the curcuminoids present in the extract of Curcuma longa is greater than the amount of any other active ingredient present in the extract of Curcuma longa. Further, the pharmacological activity and efficacy of the extract of Curcuma longa present in the composition towards alleviating the one or more symptoms associated with the medical condition is attributed to the amount of the curcuminoids present in the extract of Curcuma longa.

The extract of Zingiber officinale includes 5% to 30% of total Gingerol and Shogaol on a dry basis. The amount of the total Gingerol and Shogaol present in the extract of Zingiber officinale is greater than the amount of any other active ingredient present in the extract of Zingiber officinale. Further, the pharmacological activity and efficacy of the extract of Zingiber officinale present in the composition towards alleviating the one or more symptoms associated with the medical condition is attributed to the amount of the total Gingerol and Shogaol present in the extract of Zingiber officinale.

The method includes administering to the person an effective amount of the composition. The effective amount of the composition may range from 100 mg to 5000 mg administered to the person at one or more times in a day. Preferably, the effective amount may range from 100 mg to 2500 mg administered to the person at one or more times in a day. Still more preferably, the effective amount ranges from 500 mg to 1000 mg administered to the person at one or more times in a day. In an embodiment, the effective amount of the composition, for example, is 880 mg administered to the patient twice in a day. Further, the composition may be administered to the person suffering from the medical condition as, one or more of, but not limited to a tablet, a capsule, a suspension, and a solution. It will be appreciated by the person skilled in the art that the composition may be administered in any suitable dosage form without deviating from the scope of the invention.

In an exemplary embodiment, the method includes administering to the person the effective amount of the composition and an effective amount of one or more drugs to alleviate one or more symptoms associated with the medical condition. The one or more drugs may include, for example, but not limited to, a cyclooxygenase inhibitor, a non-steroidal anti-inflammatory drug, a disease-modifying anti-rheumatic drug, and a dietary supplement. The one or more drugs may further include one or more of a chemically synthesized drug and a drug obtained from natural source. Still further, the one or more drugs may include, for example, but are not limited to, NSAIDs, DMARDs, COX inhibitors, TNF inhibitors, corticosteroids, immuno-modulators, immuno-supressives, dietary supplement and the like.

### Example 1

The composition containing an extract of Withania somnifera, an extract of Boswellia serrata, an extract of Curcuma longa, and an extract of Zingiber officinale was prepared as a capsule. The composition was standardized as mentioned below.

The capsule:

The capsule containing 88 mg to 102 mg of the extract of Withania somnifera, 88 mg to 102 mg of the extract of Boswellia serrata, 18 mg to 27 mg of the extract of Curcuma longa, and 17 mg to 25 mg of extract of Zingiber officinale was prepared. One or two capsules may be given 2 to 3 times per day to a patient, preferably after meals.

Standardization of Raw Materials:

Specifications were developed for assessment of quality of raw materials from which the extract of Withania somnifera, the extract of Boswellia serrata, the extract of Curcuma longa, and the extract of Zingiber officinale were obtained. Thin Layer Chromatography (TLC) was used for assessment of quality of all the raw materials. Additionally, High Performance Thin Layer Chromatography (HPTLC), High Performance Liquid Chromatography (HPLC) and potentiometric techniques were also used as per requirement. The processes for extraction were also developed in the laboratory, which were later scaled up by the vendors who supplied the raw materials to the laboratory.

Standardization of Finished Composition.

The standardization of the finished composition was done using potentiometric techniques, HPTLC and HPLC assay procedures for quantification of the extract of Withania somnifera, the extract of Boswellia serrata, the extract of Curcuma longa, and the extract of Zingiber officinale present in the finished composition. The standardization of the finished compositions was confirmed by analyzing several batches of the finished compositions and the extract of Withania somnifera, the extract of Boswellia serrata, the extract of Curcuma longa, and the extract of Zingiber officinale and the finished compositions.

Silica gel was used as a stationary phase unless and otherwise mentioned. The Silica gel refers to pre-coated Silica gel 60 F254 (Merck catalogue No. 1.05554). Sample solutions were applied to TLC plates using Camag Linomat IV sample applicator. The TLC plates were scanned using Camag Scanner-3 UV/Visible densitometer. All the solvents used were LR grade solvents.

Specifications of the Extract of Withania somnifera:

The extract of Withania somnifera was a brown colored powder with a characteristic odor. The moisture content in the extract of Withania somnifera as determined by Karl Fisher titration was found to be not more than 5%. The TLC pattern was determined using a mobile phase that constituted Dichloromethane:Hexane:Methanol in a ratio of 30:20:2. The sample was prepared as 50 mg/ml of the extract of Withania somnifera in Methanol. The TLC Plate was dipped in vanillin reagent and heated at 105.degree. C. for 10 min. The characteristic Rf values were found to be .about.0.25 and 0.45. The extract of Withania somnifera was found to contain not less than 0.9% of Withanolide-D on dry basis. The total viable microbial count of the extract of Withania somnifera was also determined and found to be not more than 500 CFU/gm. The pathogens like E. coli and Salmonella were found to be absent in the extract of Withania somnifera.

Specifications of the Extract of Boswellia serrata:

The extract of Boswellia serrata was a buff colored homogeneous free-flowing powder with a characteristic odor. The extract of Boswellia serrata was prepared from oleo-gum resin of plant Boswellia serrata. The extract of Boswellia serrata was found to contain not less than 40% of total boswellic acids on dry basis. The total viable microbial count of the extract of Boswellia serrata was also determined and found to be not more than 500 CFU/gm. The pathogens like E. coli and Salmonella were found to be absent in the extract of Boswellia serrata.

Specifications of the Extract of Curcuma longa:

The extract of Curcuma longa extract was an orange-yellow colored, viscous extract of turmeric rhizomes of the plant Curcuma longa, with pungent taste and characteristic odor. The extract of Curcuma longa was found to contain not less than 27% of Curcuminoids on dry basis. The total viable microbial count of the extract of Curcuma longa was also determined and found to be not more than 500 CFU/gm. The pathogens like E. coli and Salmonella were found to be absent in the extract of Curcuma longa.

Specifications of the Extract of Zingiber officinale:

The extract of Zingiber officinale extract was a dark brown colored viscous, homogeneous extract with a characteristic odor and pungent taste. The extract of Zingiber officinale was prepared from rhizomes of plant Zingiber officinale. The extract of Zingiber officinale was found to contain not less than 14.0% of total of Gingerol and Shogaol on dry basis. The total viable microbial count of the extract of Zingiber officinale was also determined and found to be not more than 500 CFU/gm. The pathogens like E. coli and Salmonella were found to be absent in the extract of Zingiber officinale.

### Example 2

### Carrageenan-Induced Paw Edema in Rats

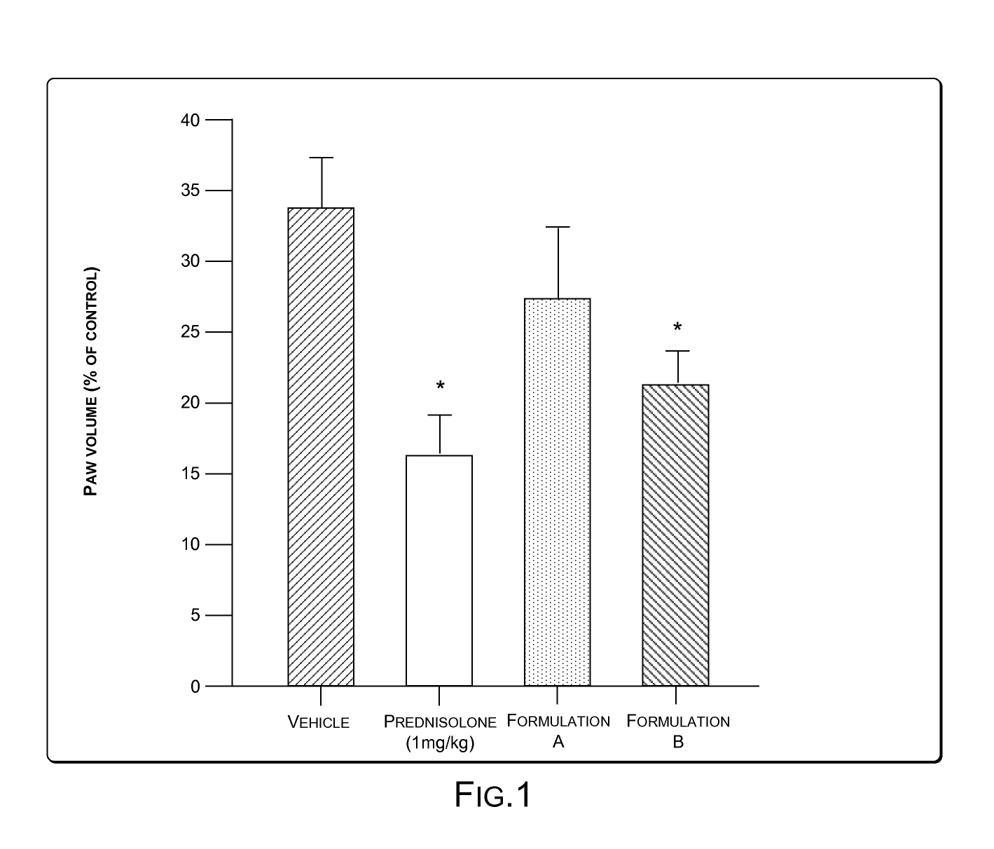

A study was conducted to determine effectiveness of the composition in inhibiting carrageenan-induced paw edema (COX-2 mediated) in rats. The composition was prepared as a Formulation (herein after, Formulation B). Formulation B was prepared according to the specifications mentioned in Example 1. Formulation B contained not less than 0.9% Withanolide-D on dry basis in addition to other constituents. Another formulation, Formulation A was prepared such that Formulation A had lesser concentration of Withanolide-D as compared to the concentration of Withanolide-D in Formulation B. A standard solution of Carrageenan, an inflammatory agent was injected in the paw of rats (animal) to produce swelling. The swelling was measured by a Plethysmograph, which is an instrument that measures the extent of paw swelling due to injection of Carrageenan. The animals were labeled as Vehicle, Prednisolone (as positive control), Formulation A, and Formulation B. The effectiveness of the Vehicle, Prednisolone, Formulation A, and Formulation B in controlling the swelling were evaluated separately and compared and the results were recorded. FIG. 1 illustrates a comparison between anti-inflammatory activities of Vehicle, Prednisolone, Formulation A, and Formulation B in carrageenan-induced paw edema in rats. It was found that significant anti-inflammatory activity was exhibited by the Formulation B compared to Formulation A that contained less Withanolide-D. However, it was also concluded that, Formulation A exhibited anti-inflammatory activities that was less robust as compared to the anti-inflammatory activity exhibited by the Formulation B.

### Example 3

### Granuloma Pouch Assay in Rat

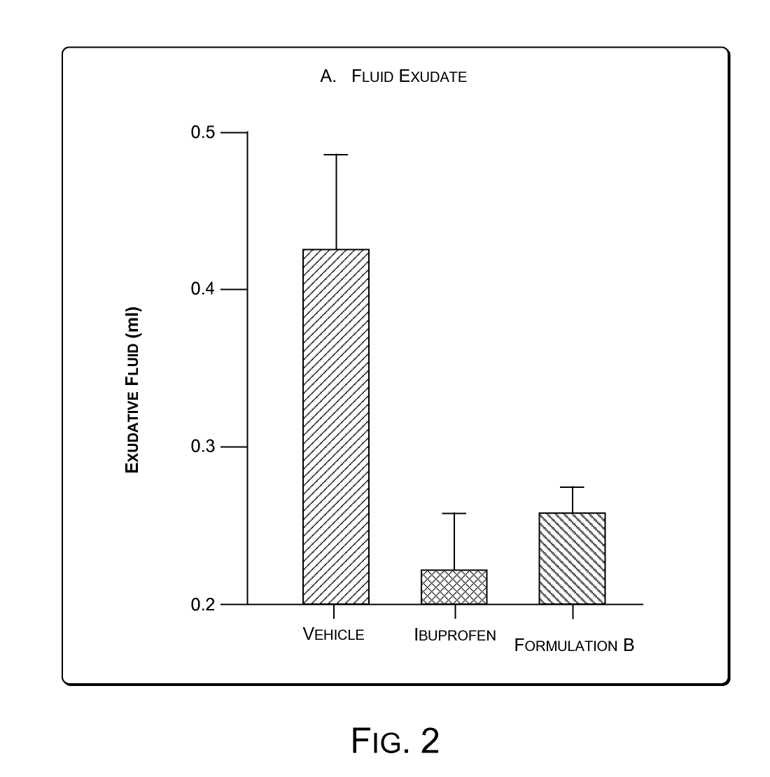

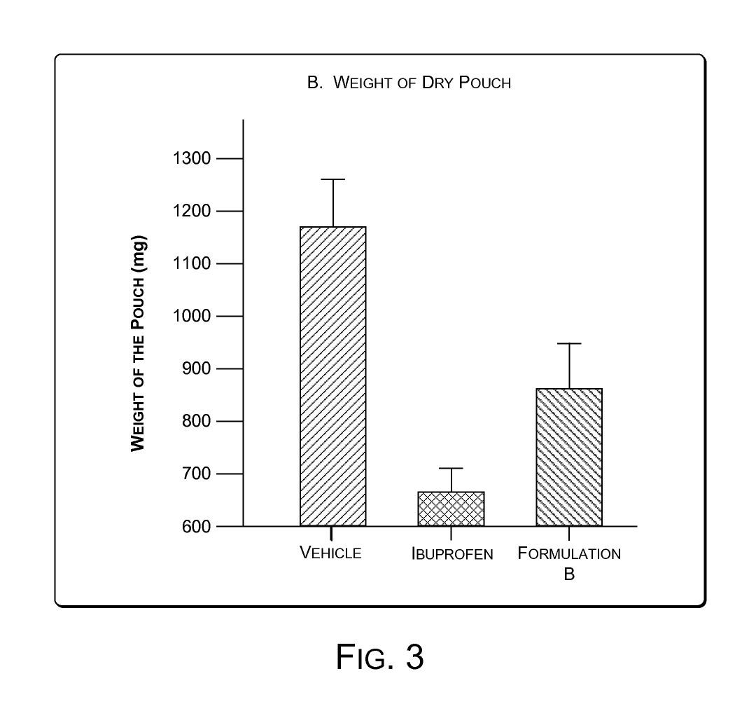

Granuloma represents the exudative and proliferative phase of inflammation in croton oil-induced inflammation. Croton oil induces some surge of Interleukin 1.beta.(IL-1.beta.) and Myeloperoxidase (MPO). IL-1.beta. and MPO are markers of cutaneous inflammation. A significant inflammatory condition was developed as a granuloma pouch containing exudative fluid over a period of 4-8 days in rats (animals). The animals were labeled as Vehicle, Ibuprofen and Formulation B. Anti-inflammatory drugs i.e. Ibuprofen and Formulation B were given to correspondingly labeled animals daily for 4-8 days to inhibit the formation of the exudative fluid. The change in the volume of the exudative fluid in the vehicle and in animals treated with Ibuprofen and Formulation B was measured. FIG. 2 illustrates change in the volume of the exudative fluid in the vehicle and in animals treated with Ibuprofen and Formulation B. The Formulation B was found to show significant anti-inflammatory activity. After removing the exudative fluid from the granuloma pouches, the granuloma pouches were dried. The change in the dry weight of the granuloma pouches in the vehicle and animals treated with Ibuprofen and Formulation B was measured. FIG. 3 illustrates change in the weight of dried granuloma pouches in the vehicle and in animals treated with Ibuprofen and Formulation B. Significant anti-inflammatory activity was exhibited by Formulation B.

### Example 4

### Adjuvant Induced Arthritis in Rats

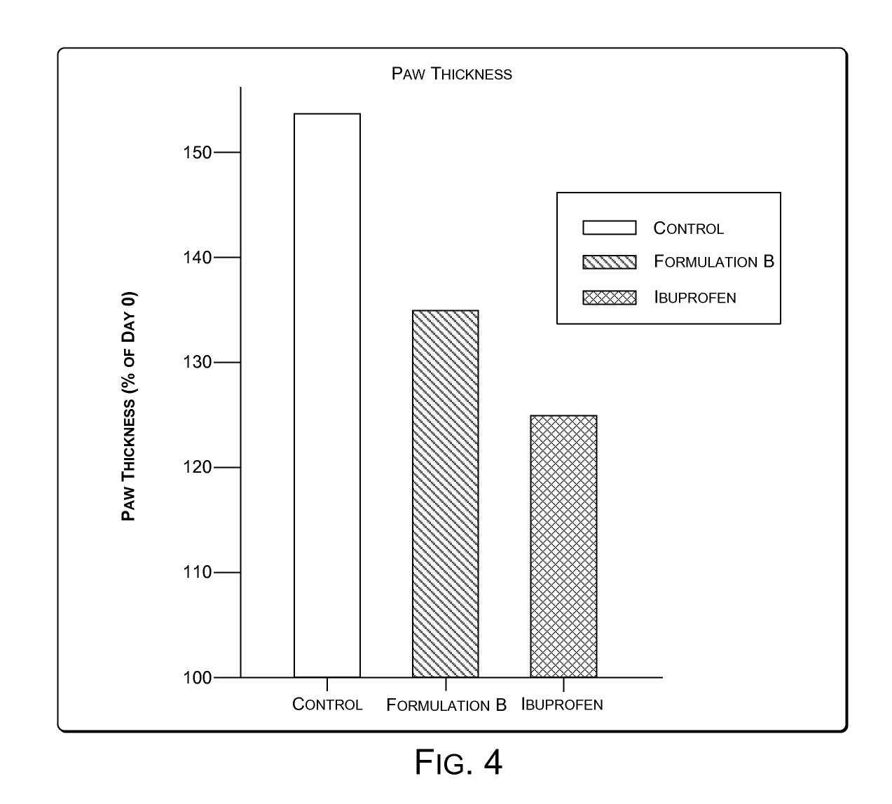

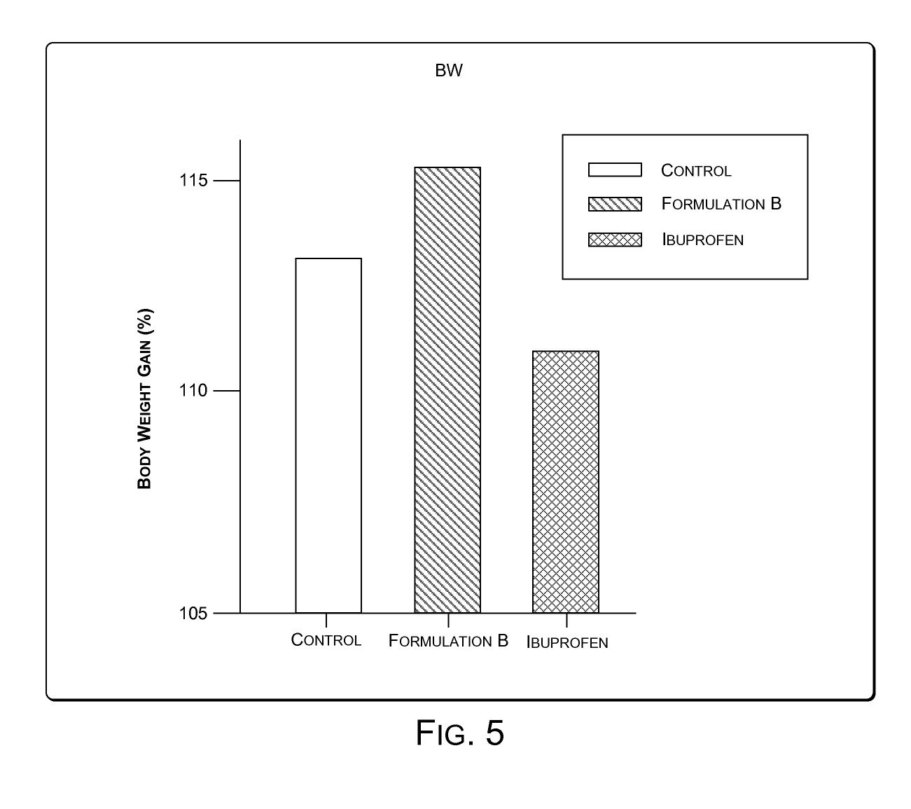

Arthritis or inflammation in the joint was induced by injection of Complete Freund's Adjuvant (CFA) into the left hind footpad of rats (animals). The animals were divided into three groups namely, Control, Ibuprofen and Formulation B. The role of Formulation B on specific cytokine blockade in the etiology of cachexia caused by Adjuvant Arthritis (AA) was evaluated. The parameter considered for the evaluation included paw thickness as a percent of paw thickness at Day 0 and percent body weight gain in animals. FIG. 4 illustrates paw thickness in animals as a percent of the paw thickness at Day 0 during the Adjuvant Induced Arthritis study in animals. Whereas, FIG. 5 illustrates percent body weight gain in animals during the Adjuvant Induced Arthritis study in animals. It was found that treatment of Ibuprofen did not show any effect on cytokine inhibition, and animals treated with Ibuprofen lost body weight significantly as compared to the control animals. However, inhibition of other cytokines, probably one or more of TNF-.alpha., IL-6, and IL-1.beta. are needed to mitigate AA-associated weight loss, as demonstrated by the Formulation B. This weight gain effect is dissociated from the effect of such inhibition on joint inflammation by Formulation B and Ibuprofen (FIG. 4).

### Example 5

### PDE-4 Enzyme Assay

PDE-4 partially purified from human U-937 myeloid leukemia cells was used. Test Formulation B and vehicle was incubated with 0.2.mu.g enzyme and 1.mu.M cAMP containing 0.01.mu.M [3H]cAMP in Tris buffer pH 7.5 for 20 minutes at 25.degree. C. The reaction was terminated by boiling for 2 minutes and the resulting AMP was converted to adenosine by addition of 10 mg/ml snake venom nucleotidase and further incubation at 37.degree. C. for 10 minutes. Unhydrolyzed cAMP was bound to AG1-X2 resin, and remaining [3H]Adenosine in the aqueous phase was quantitated by scintillation counting. The said Formulation B at 5.mu.g/ml inhibited 41% of the enzyme.

### Example 6

### Chronic Oral Toxicity

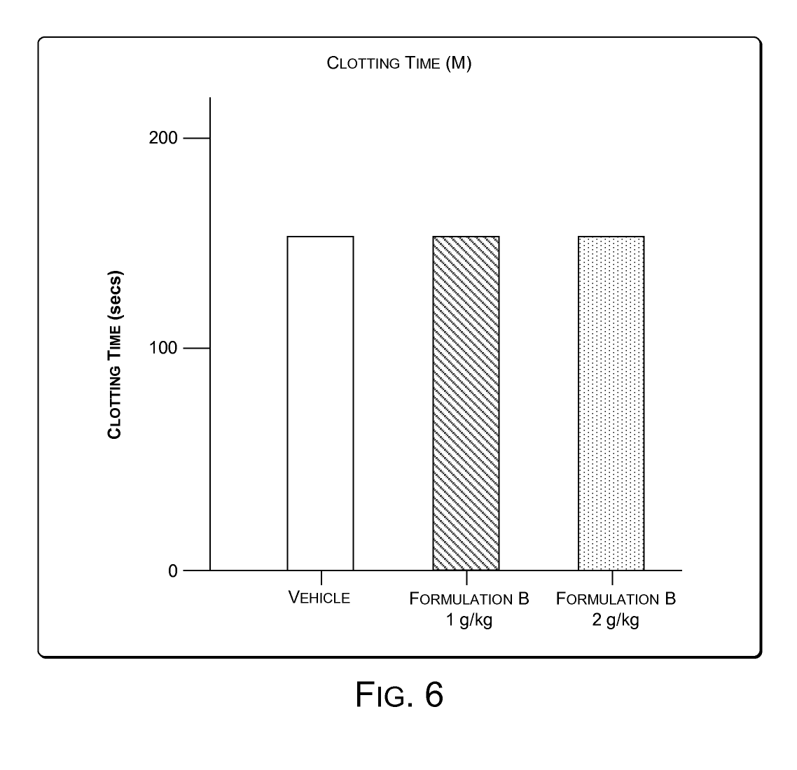

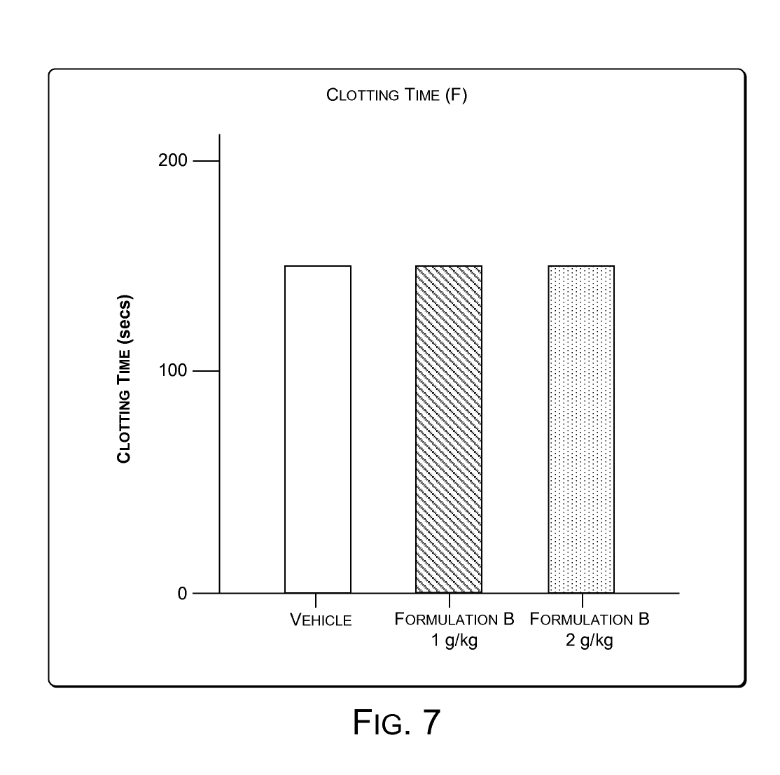

Two doses were selected for chronic (180 days) toxicity study in rats. These doses were 1 g/Kg body weight and 2 g/Kg body weight. The purpose of this study was to assess the toxicological profile of Formulation B when administered orally at the dose levels of 1 g/Kg body weight and 2 g/Kg body weight to Norwegian strain albino rats (20 males and 20 females per dose) for 180 days. The results were recorded. FIG. 6 illustrates the clotting time in seconds in the animals (male) over a period of 180 days of toxicity study using Formulation B. FIG. 7 illustrates the clotting time in seconds in the animals (Female) over a period of 180 days of toxicity study using Formulation B. It was observed that unlike other NSAIDs, Formulation B did not change the clotting time in the animals after 180 days of the toxicity study.

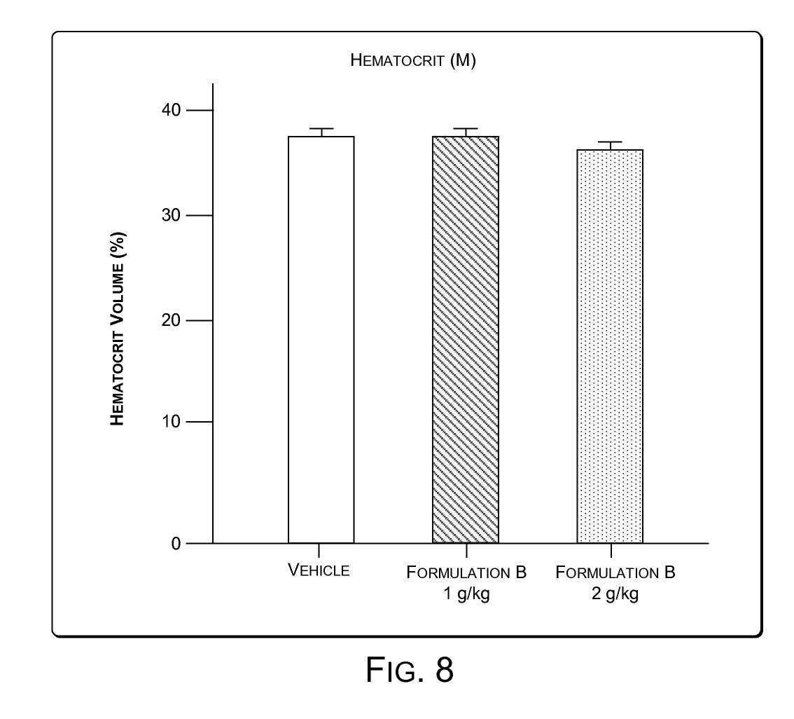

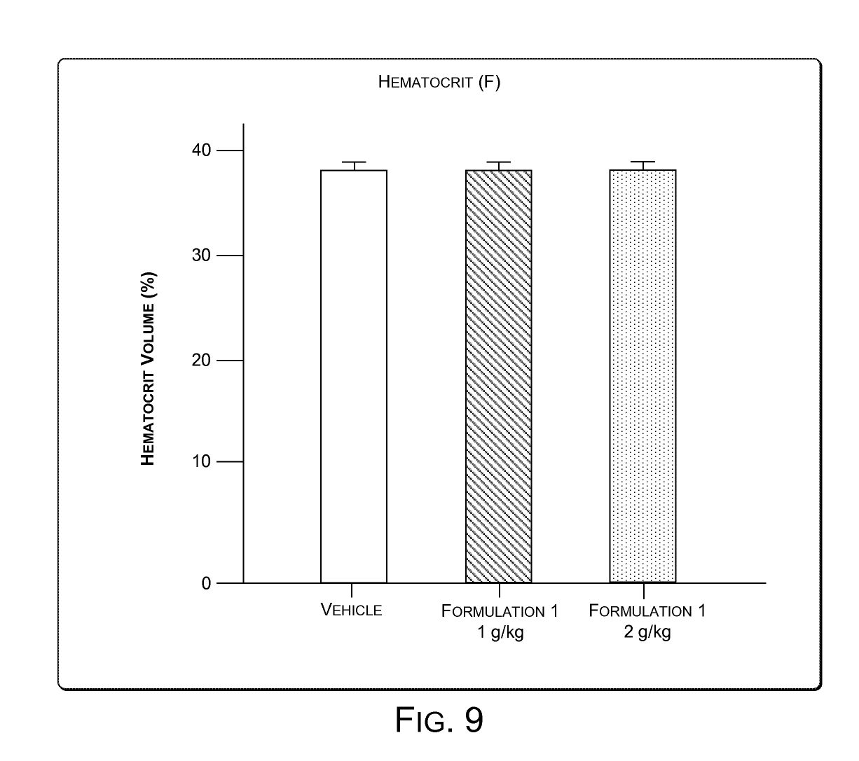

Further, FIG. 8 illustrates the percent Hematocrit Volumes of the animals (Male) over a period of 180 days of toxicity study using Formulation B. FIG. 9 illustrates the percent Hematocrit Volumes of the animals (Female) over a period of 180 days of toxicity study using Formulation B. It was found that the plasma volume of the animals remained unchanged in the animals after 180 days of the toxicity study. Ulcerogenic potential was also checked and there were no gross changes in gastrointestinal wall after the treating the animals with Formulation B for 180 days. The results clearly differentiate that the activity of Formulation B is not merely because of cyclooxygenase inhibition.

### Example 7

### Cytochrome P450 (CYP450) Studies

CYP450 assays are generally designed to predict potential drug-drug interactions for screening new drugs/chemical entities.

Human recombinant CYP450 3A4, CYP450 2c9, CYP450 2c19, expressed in baculovirus infected insect cells were incubated with 0.5.mu.g/ml of Formulation B and the vehicle preincubated with 0.8 pmole enzyme for 15 minutes at 37.degree. C. The reaction was initiated by addition of 50.mu.M 7-benzyloxy-4-(trifluoromethyl)-coumarin, cofactor solution (1.3 mM NADP, 3.5 mM glucose-6-phosphate, 3.3 mM MgCl2 and 0.4 U/ml G6P dehydrogenase in phosphate buffer pH 7.5) for 30 minutes and terminated by further addition of 80% acetonitrile. No significant inhibition of any of the tested CYPs i.e. CYP450 3A4, CYP450 2c9, CYP450 2c19 was observed. Thus, the Formulation B was found to be generally devoid of potential drug-drug interactions.

### Example 8

### Potassium Channel hERG (Human)

The hERG potassium channel is a major molecular component of the delayed rectifier current (I.sub.Kr) underlying cardiac repolarization. Due to either genetic defects in its pore-forming subunit or adverse drug effects, decreased hERG activity prolongs the QT interval and can lead to the potentially lethal ventricular arrhythmia Torsades de pointes. The implication of (I.sub.Kr) in cardiac arrhythmias and in anti-arrhythmic/pro-arrhythmic actions of drugs has driven intensive research interests in its structure-function relationship, the linkage between LQT-associated mutations and changes in channel function, and the mechanism of drug actions.

The assay to measure binding of [.sup.3H]Astemizole to potassium channel hERG was conducted in order to determine the inhibitory effect of Formulation B on the potassium channel hERG. HEK-293 cells stably transfected with a plasmid encoding the human potassium channel hERG were used to prepare modified HEPES pH 7.4 buffer using standard techniques. A 10.mu.g aliquot of membrane was incubated with 1.5 nM [.sup.3H]Astemizole for 60 minutes at 25.degree. C. in presence of 0.5.mu.g/ml of the Formulation B. Non-specific binding was estimated in the presence of 10.mu.M Astemizole. Membranes were filtered and washed 3 times and the filters were counted to determine [.sup.3H]Astemizole specifically bound to hERG. It was concluded that the Formulation B did not inhibit the potassium channel hERG.

### Example 9

### Anti-Inflammatory Activities of Celecoxib, Chondroitin and the Formulation B in Experimental Animal Models

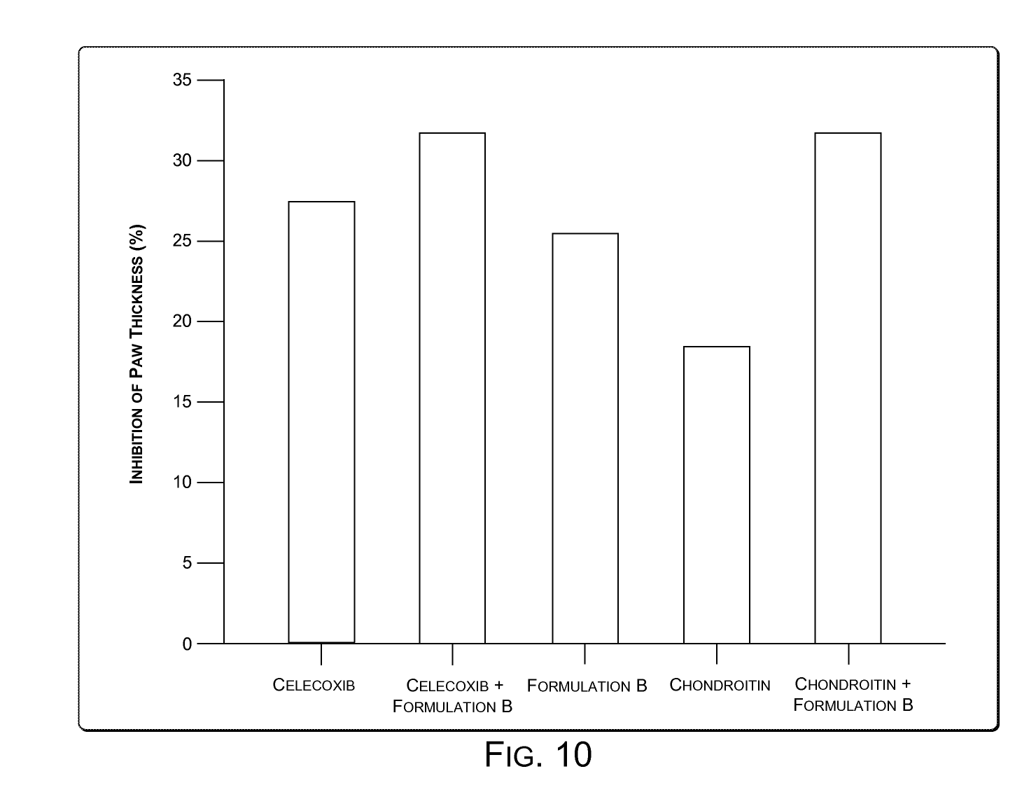

Anti-inflammatory activities of Celecoxib; Celecoxib in combination with Formulation B; Formulation B; Chondroitin; Chondroitin in combination with Formulation B were evaluated in carrageenan-induced rat paw edema. 0.1 ml of 1% WN carrageenan (Ottokemi, B. No. C 1675) solution injected intradermally in the right paw of the rat. Whereas, the left paw was not injected with carrageenan so that the left paw can serve as control. Following drugs were given to the animals separately: A. 1 mg/Kg body weight of Celecoxib; B. 166 mg/Kg body weight of Chondroitin; C. 1000 mg/Kg body weight of the Formulation B; D. a combination of Celecoxib and Formulation B; and E. a combination of Chondroitin and Formulation B. The results were recorded. FIG. 10 illustrates the anti-inflammatory activities of Formulation B; Celecoxib; Celecoxib in combination with Formulation B; Chondroitin; and Chondroitin in combination with Formulation B. Significant anti-inflammatory activity was exhibited by Formulation B. Further, Formulation B was found to improve anti-inflammatory activity of Celecoxib and Chondroitin.

### Example 10

### Inhibition of TNF-.alpha. and iNOS in LPS Induced Macrophages

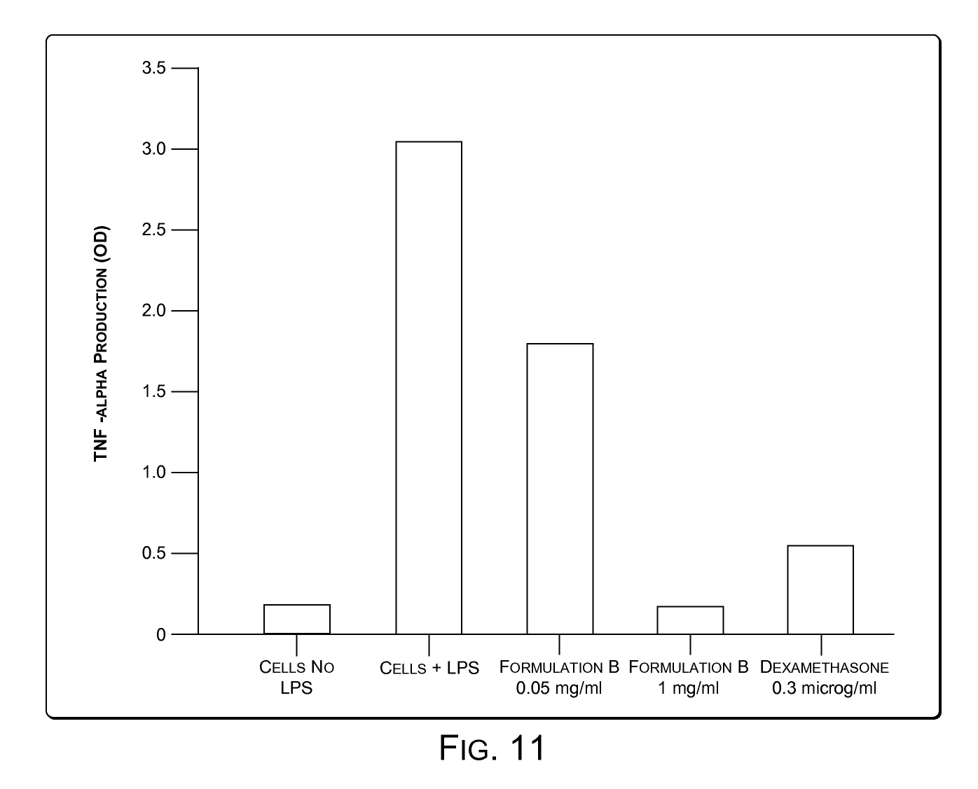

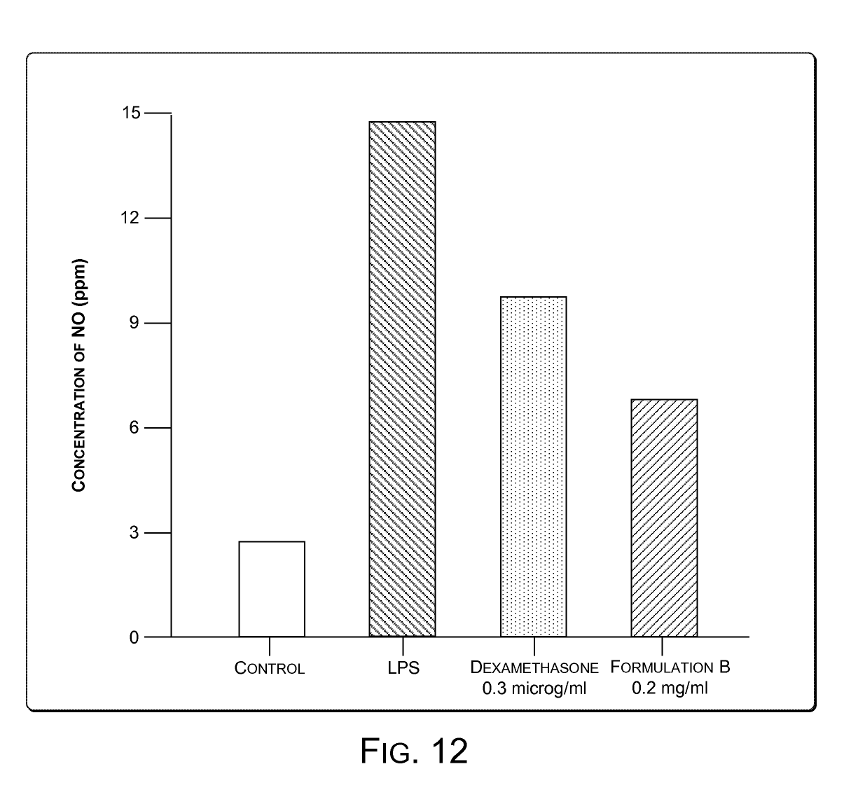

Mouse macrophage cells were plated in 96 well plates. Thereafter, the mouse macrophage cells were preincubated with Formulation B and Dexamethasone (as positive control) separately for an hour. Subsequently, the mouse macrophage cells were challenged with LPS for 24 hours. The levels of TNF-.alpha. and Nitric Oxide (NO) produced in the mouse macrophage cells activated by LPS were measured by ELISA method. FIG. 11 illustrates production of TNF-.alpha. in the mouse macrophage cells activated by LPS and treated separately with Dexamethasone and different doses of Formulation B. Whereas, FIG. 12 illustrates production of NO in the mouse macrophage cells activated by LPS and treated separately with Dexamethasone and different doses of Formulation B. It was found that Formulation B significantly inhibited production of TNF-.alpha. and NO at a dose of 0.1 mg/ml.

### Example 11

### Dextran Sodium Sulfate (DSS) Induced Colitis

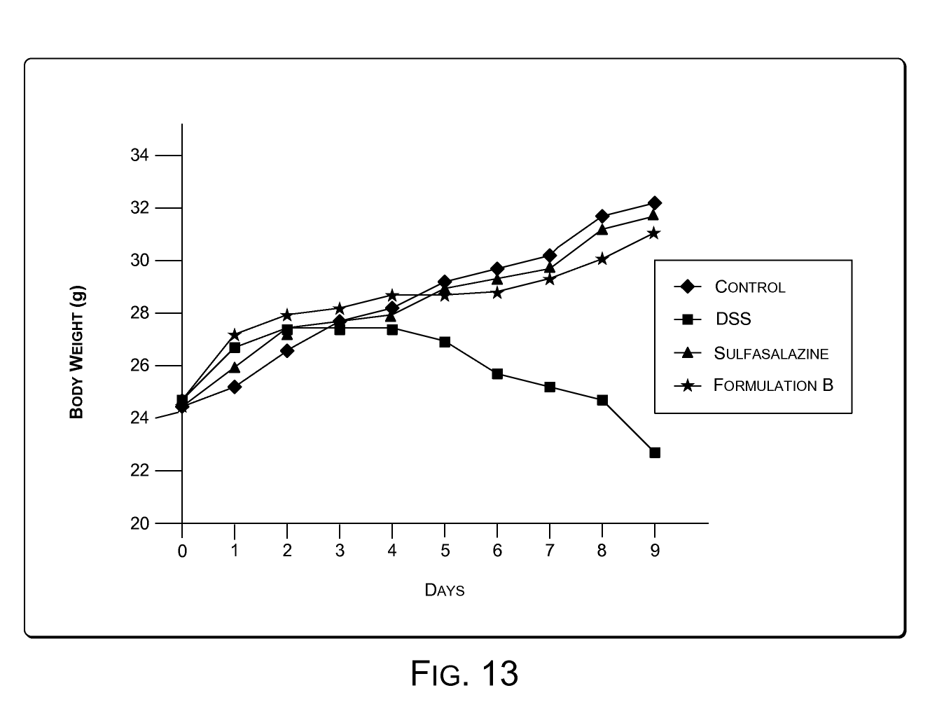

Inflammation was induced in mice (animal) using oral administration of DSS. Weight loss was noted in the control animals with time. Treatment with either Formulation B or Sulfasalazine improved the body weight loss. Food intake in animals was also reduced with time upon oral administration of DSS. The reduction in food intake was also recovered upon treatment of animals with Formulation B or Sulfasalazine. FIG. 13 illustrates effectiveness of Formulation B and Sulfasalazine in inhibiting DSS induced colitis, wherein the inhibition of DSS induced colitis is measured as improvement in body weight loss in animals. It was found that Formulation B has almost same effectiveness in inhibiting DSS induced colitis in mice as compared with Sulfasalazine.

Various embodiments of the present invention provide method and compositions for alleviating one or more symptoms associated with the medical conditions mediated by one or more of pathological mediators like, cyclooxygenases, pro-inflammatory cytokines, and pro-inflammatory enzymes. The composition includes the extract of Withania somnifera, the extract of Zingiber officinalis, the extract of Curcuma longa and the extract of Boswellia serrata that are purified, optimized and concentrated plant extracts, as shown in Example 1. The methods and compositions in accordance with the invention provide for alleviating pain and inflammation associated with various medical conditions.

The compositions in accordance with the invention are generally devoid of side-effects, toxicity and risks that are associated with the conventional anti-inflammatory drugs like, NSAIDs, DMARDs, COX inhibitors, TNF inhibitors, corticosteroids, immune-modulators, immuno-supressives, and the like. In addition, the compositions are substantially devoid of drug-drug interactions and hence can be safely used in combination therapy for inflammations along with known anti-inflammatory drugs. Further, the compositions in accordance with the invention provide for synergistic anti-inflammatory effects when used with known anti-inflammatory drugs.

Those skilled in the art will realize that the above-recognized advantages and other advantages described herein are merely exemplary and are not meant to be a complete rendering of all of the advantages of the various embodiments of the present invention.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of the present invention. The benefits, advantages, solutions to problems, and any element(s) that may cause any benefit, advantage, or solution to occur or become more pronounced are not to be construed as a critical, required, or essential features or elements of any or all the claims. The present invention is defined solely by the appended claims including any amendments made during the pendency of this application and all equivalents of those claims as issued.

1. A method for alleviating at least one symptom associated with a medical condition mediated by at least one of a cyclooxygenase, a pro-inflammatory cytokine, and a pro-inflammatory enzyme, the method comprising:
administering to a person suffering from the medical condition a therapeutic dose of an agent selected from the group consisting of celecoxib, dexamethasone, and sulfasalazine; and
administering to the person suffering from the medical condition a composition comprising a set of plant extracts, wherein the set of plant extracts comprises an extract of Withania somnifera, an extract of Boswellia serrata, an extract of Curcuma longa, and an extract of Zingiber officinale.

2. (canceled)

3. The method of claim 1, wherein the at least one extract comprises the extract of Withania somnifera, wherein the at least one desired active ingredient comprises Withanolide-D, and wherein an amount of Withanolide-D present in the extract of Withania somnifera is greater than an amount of other active ingredients present in the extract of Withania somnifera.

4. The method of claim 1, wherein the set of plant extracts comprises 10% to 75% of the extract of Withania somnifera, 10% to 75% of the extract of Boswellia serrata, 3% to 30% of the extract of Curcuma longa, and 3% to 25% extract of Zingiber officinale.

5. The method of claim 1, wherein the set of plant extracts comprises the extract of Withania somnifera, the extract of Boswellia serrata, the extract of Curcuma longa, and the extract of Zingiber officinale in a concentration ratio of 1:1:0.25:0.2 respectively.

6. (canceled)

7. The method of claim 1, wherein the effective amount of the composition ranges from 100 mg to 2500 mg.

8. The method of claim 1, wherein the composition is selected from a group comprising a tablet, a capsule, a powder, a suspension, and a solution.

9. The method of claim 1, wherein the cyclooxygenase is cyclooxygenase-2 (COX-2).

10. The method of claim 1, wherein the pro-inflammatory cytokine is at least one of Tumor Necrosis Factor-.alpha. (TNF-.alpha.), Interleukin-6 (IL-6), and Interleukin-1 (IL-1).

11. The method of claim 1, wherein the pro-inflammatory enzyme is at least one of phosphodiesterase-4 (PDE-4) and an Inducible Nitric Oxide Synthetase.

12. The method of claim 1, wherein the medical condition is selected from the group consisting of a rheumatoid arthritis, an inflammatory bowel disease, Crohn's disease, a psoriasis, and an ankylosing spondylitis.
